(19) United States
(12) Patent Application Publication
(10) Pub. No.: US 2021/0000426 A1
(43) Pub. Date: Jan. 7, 2021

(54) CLASSIFICATION SYSTEM OF EPILEPTIC EEG SIGNALS BASED ON NON-LINEAR DYNAMICS FEATURES

(71) Applicant: Peking University, Beijing (CN)

(72) Inventors: Shan'en CHEN, Beijing (CN); Xi ZHANG, Beijing (CN)

(73) Assignee: Peking University, Beijing (CN)

(21) Appl. No.: 16/820,865

(22) Filed: Mar. 17, 2020

(30) Foreign Application Priority Data
Jul. 4, 2019 (CN) 201910597746.8

(51) Int. Cl. A61B 5/00; A61B 5/04; G06K 9/46; G06K 9/62

(52) U.S. Cl. CPC A61B 5/7267; A61B 5/04004; A61B 5/726; G06K 9/623; G06K 9/46; A61B 5/7203; A61B 5/4094

(57) ABSTRACT

A classification system of epileptic EEG signals based on non-linear dynamics features includes a preprocessing module, a feature extraction module, a feature sorting module, a feature selection module and a classification module: the preprocessing module uses discrete wavelet transformation to remove noise in the EEG data and obtain effective EEG signal data without noise; the feature extraction module uses multiple entropy algorithms to calculate the non-linear dynamics features of each EEG signal; the feature sorting module sorts features with analysis of variance; the feature selection module selects the optimal feature subset that has the most significant impact on the accuracy of the model uses a uses a forward sequential feature selection algorithm; the classification module transforms the judgment of EEG during the period of epilepsy and EEG during the interval period of epilepsy into a binary classification problem by use of a least squares support vector machine algorithm.

### CROSS REFERENCE TO RELATED APPLICATIONS

Applicant claims priority under 35 U.S.C. § 119 of Chinese Application No. 201910597746 .8 filed Jul. 4, 2019, the disclosure of which is incorporated by reference.

### BACKGROUND OF THE INVENTION

### 1. Field of the Invention

The disclosure relates to a classification system of epileptic EEG signals based on non-linear dynamics features, in particular to a system that uses multiple entropies to extract the non-linear dynamics features of EEG to classify epileptic EEG signals, and belongs to the field of neural information technology.

### 2. Description of the Related Art

Epilepsy is a common and multiple chronic neurological disease, and epileptic seizures are caused by irregular neurons and irregular discharges of neurons, which are caused by synchronous or excessive activity of neurons in the brain. During epileptic seizures, it will cause dysfunction of movement, behavior, consciousness and sensation. Therefore, epileptic seizures may lead to various fatal consequences. Around the world, over 50 million people suffer from epilepsy, and over 200,000 new cases are diagnosed every year. The treatment methods for epileptic seizures include surgery, drugs, galvanic stimulation and so on. Before determining the treatment, the most important thing is to detect patients with suspected epilepsy. At present, the general method for seizure detection of epileptic activity is based on visual inspection of huge amounts of electroencephalography (EEG) signals by doctors. Since the patient's EEG signals need to be detected and classified for a long time, the traditional doctor detection method is very time-consuming and labor-intensive. Many hospitals even delay the best treatment time for patients due to the slow detection speed caused by the lack of relevant doctors. On the other hand, since the traditional epilepsy detection relies on the doctor's visual observation and subjective judgment for classification, sometimes it is easy to make mistakes, which may lead to accidental misdiagnosis. Therefore, there is an urgent need to develop an automatic classification system of epileptic EEG signals to lighten the workload of doctors and also to reduce misdiagnosis caused by errors in visual inspection. Therefore, the classification and detection of EEG signals in epileptic seizures has important clinical application value.

EEG is widely used in epilepsy detection and analysis. Human EEG signals are formed by the interaction of hundreds of millions of neurons, so they have the characteristics of time-varying, nonlinearity and instability. At the same time, EEG data signal will produce random error after measurement, and EEG signal will also be affected by individual differences. Therefore, the analysis of EEG data signal becomes a difficult problem. There are many kinds of early warning methods for epileptic signals, however, various algorithms have a variety of shortcomings in the accuracy, sensitivity and specificity because of the complexity of epileptic EEG signal itself, and for example, if the accuracy is high, the specificity will be reduced.

At present, the existing methods of EEG signal analysis include time-domain and frequency-domain analysis and probability statistical analysis, but none of these methods can capture the nonlinear characteristics of the signal well. The theory of nonlinear dynamics is applied to a variety of signal processing scenarios including the processing of EEG signals. As a kind of non-linear dynamic index, entropy reflects the degree of chaos in the system and can reveal the chaotic behavior of brain. In recent years, the entropy has been widely used in EEG signal analysis. Researchers have proposed different entropy concepts in different fields, such as Sample Entropy, Conditional Entropy and Spectral Entropy. Different entropies can reflect different nonlinear characteristics of the system. However, in the field of epileptic EEG signal analysis, most studies use a single entropy to measure the characteristics of EEG, which cannot cover most of the characteristics of epilepsy EEG, resulting in shortcomings in the accuracy, sensitivity and specificity of various algorithms There is a lack of a method for fusion of different entropies to extract the characteristics of epileptic EEG, and a large number of nonlinear dynamic features contained in EEG signals cannot be fully characterized.

### SUMMARY OF THE INVENTION

In view of shortages in the prior art, the present invention provides a system that uses multiple entropies to extract the non-linear dynamics features of EEG to classify epileptic EEG signals. The system can fully extract the non-linear dynamics features of EEG signals in different states, fully reflect the state changes of brain nerve discharge, and capture the epileptic moment, thereby improving the classification accuracy of EEG signals.

The object of the present invention is achieved by the following technical solutions.

A classification system of epileptic EEG signals based on non-linear dynamics features includes a preprocessing module, a feature extraction module, a feature sorting module, a feature selection module, and a classification module. The five modules are connected in order to train the classification model according to an EEG database. The specific technical solutions are provided as follows.

Firstly, the EEG signals are preprocessed one by one. The preprocessing module uses Discrete Wavelet Transformation (DWT) to remove noise in the EEG data and obtain effective EEG signal data without noise.

The feature extraction module first divides the EEG signals into several data segments, and uses multiple entropy algorithms to calculate different entropy values of EEG data under the same time window as the characteristic values of the corresponding data segments. A feature set is formed by calculating the entropy values of all entropy algorithms

The feature sorting module sorts the significant influence of the nonlinear dynamic characteristics of the extracted EEG signals on the classification results of epileptic EEG signals by one-way analysis of variance (ANOVA). The more significant the influence of feature variables on classification results, the higher the sorting of the feature variables.

The feature selection module uses a forward sequential feature selection (FSFS) algorithm to successively add one feature from the first most significant feature into the classification model until the accuracy of the model is no longer improved, so as to select the optimal feature subset that has the most significant impact on the accuracy of the model.

The classification module transforms the judgment of epileptic seizure state into a binary classification problem by use of a least squares support vector machine (LS-SVM) algorithm, and classifies the EEG signals of epileptic patients, and the collected EEG signals are used as the training data of LS-SVM to train the classification model. After offline training of the above process, the hyper-parameters of the LS-SVM are obtained, and an optimized feature subset is selected.

As a preferred solution, the discrete wavelet transform method that is used for EEG signal denoising in the pre-processing module is to use a Daubeches-4 wavelet function, and select an EEG signal with a frequency of 3 to 25 Hz after filtering.

As a preferred solution, four entropy algorithms are used to calculate different entropy values of EEG signals in the feature extraction module.

As a preferred solution, the classification module first trains a least squares support vector machine algorithm, and the training method of the least squares support vector machine algorithm is as follows: the EEG signal database of epilepsy patients is randomly divided into two parts: 70% and 30%. 70% of the EEG data is used to train algorithm, and the remaining 30% data is used to test the algorithm so as to obtain a LS-LVM model.

The invention further increases a real-time on-line system to perform real-time online classification of new EEG signals collected in real-time through the pre-processing module, the feature extraction module (an optimized feature subset), and the classification module according to the above process.

The invention has the advantages that, the feature extraction module of the invention calculates the nonlinear dynamic characteristics of each EEG signal with a variety of entropy algorithms; the feature sorting module sorts features with analysis of variance; the feature selection module selects the optimal feature subset that has the most significant impact on the accuracy of the model uses a uses a forward sequential feature selection (FSFS) algorithm; and the classification module transforms the judgment of epileptic seizure and seizure interval into a binary classification problem by use of a least squares support vector machine (LS-SVM) algorithm, which has the advantages of low computational complexity, good real-time performance and higher accuracy, and can be used to quickly identify the feature changes of EEG signals and realize classification of epileptic EEG signals. The classification system of epileptic EEG signals based on non-linear dynamics features provided by the invention is applied to the EEG signal of epileptic patients, realizing the high accuracy, sensitivity and specificity of the classification of epileptic EEG signals.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1 is a structural block diagram of a method for detecting epilepsy according to the present invention; and

FIG. 2 shows original EEG signals during epileptic seizures and sub-signals of each frequency segment after DWT decomposition.

### DETAILED DESCRIPTION OF PREFERRED EMBODIMENTS

The invention will be described in detail in combination with the attached Drawings.

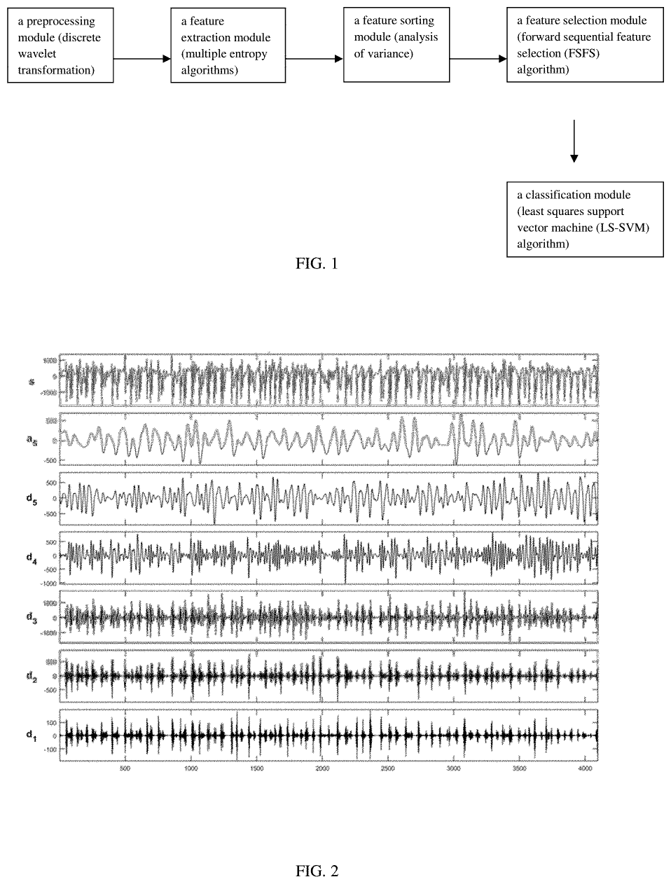

As shown in FIG. 1, the classification system of epileptic EEG signals based on non-linear dynamics features of the invention includes a preprocessing module, a feature extraction module, a feature sorting module, a feature selection module, and a classification module:

(1) Preprocessing Module

The EEG data is preprocessed. The original single channel EEG data (as shown in FIG. 2) is filtered and denoised by the Daubeches-4 wavelet function one by one. After filtering, the EEG signal with a frequency of 3 to 25 Hz is selected, that is, three sub-signals d3, d4, d5.

(2) Feature Extraction Module

Four entropy algorithms (Shannon entropy, conditional entropy, sample entropy and spectral entropy) are used to calculate the nonlinear dynamic characteristics of the three preprocessed sub-signals respectively. The calculation methods of the four entropy algorithms are given by the following formulas:

a) SHANNON ENTROPY (ShanEn)

Given a time series X={xᵢ,i=1,2, . . . ,N}, ShanEn is defined as:

ShanEn=−Σᵢ₌₁^{N}p(xᵢ)log(p(xᵢ)).

where p(xᵢ) is the probability distribution function of X with Σᵢ₌₁^{N}p (xᵢ)=1, 0≤p(xᵢ)≤1.

b) CONDITIONAL ENTROPY (CondEn)

Given a time series X={xᵢ,i=1,2, . . . ,N, CondEn can be calculated by the following two steps: Firstly, the phase space of X is reconstructed according to the sequence order, and a set of m-dimension vectors are generated, i≥m. After reconstruction, (N−m+1) new vectors are obtained: xₘ(i)={Xᵢ,xᵢ₋₁, . . . xᵢ₋ₘ₊₁}. Each Xₘ(i) vector represents a pattern of m consecutive sample points. Next, CondEn can be calculated by the following formula:

$$CondEn ( \frac{m}{m - 1} ) = - {\sum }_{m - 1} {p}_{m - 1} {\sum }_{m \vert ( m - 1 )} {p}_{m \vert ( m - 1 )} log {p}_{m \vert ( m - 1 )} ,$$

wherein, pₘ₋₁ represents the joint probability of Xₘ₋₁(i), and p_{m|(m-1}) represents the conditional probability of Xₘ(i) in the case that Xₘ₋₁(i) is given.

c) SAMPLE ENTROPY (SampEn)

Given a time series X={xᵢ,i=1,2, . . . ,N}, given threshold r and dimension m, generating a set of m-dimensional vectors: Xₘ(i)={xᵢ,xᵢ₊₁, . . . ,Xᵢ₊ₘ₋₁}, it is defined that the distance d[Xₘ(i), Xₘ(j)] between the vectors Xₘ(i) and Xₘ(j) is the one with the largest difference between the two corresponding elements, that is,

d[Xₘ(i), Xₘ(j)]=max[|x(i+k)−x(j+k)|]

wherein, m−1≥k≥0,i≠j,i≥1,N−m≥j.

For a given threshold r, when the dimension is m and m+1, the number of d[Xₘ(i),Xₘ(j)]<r is counted as B and A respectively, then the SampEn can be defined as the following formula:

$$S a m p E n = - log \frac{A}{B} .$$

d) SPECTRAL ENTROPY (SE)

SE is often used to measure the disorder degree of signals in the frequency distribution of amplitude component of signal power spectrum. When the signal centralizes in one frequency, the spectral entropy se reaches the minimum value. SE can be defined by:

SE=−Σ_{ƒ}p_{ƒ}log(p_{ƒ}),

where ƒ is the frequency, and p_{ƒ}is the power spectral density at frequency ƒ obtained from Fourier transform.

The above four entropies are calculated for all three decomposed EEG signals. Finally, each EEG signal segment has a total of 3*4 entropy features, which are input into the feature sorting module for weight sorting.

(3) Feature Sorting Module

The feature sorting module sorts the significant influence of the nonlinear dynamic characteristics (4 types, 12 entropy values) of the extracted EEG signals on the classification results of epileptic EEG signals by one-way analysis of variance (ANOVA). The more significant the influence of feature variables on classification results, the higher the sorting of the feature variables. The 12 features processed by the sorting module are input into the feature selection module to select the features that have significant influence on the classification results.

(4) Feature Selection Module

The feature selection module of the invention uses a forward sequential feature selection (FSFS) algorithm to successively add one feature from the first most significant feature into the classification model until the accuracy of the model is no longer improved, so as to select the optimal feature subset that has the most significant impact on the accuracy of the model.

(5) Classification Module

The classification module of the invention determines the seizure state of EEG signals by use of a least squares support vector machine (LS-SVM) algorithm. The least squares support vector machine (LS-SVM) is an improved support vector machine, which overcomes the shortcomings of high computational burden of support vector machines, has stronger real-time performance and is often used to recognize and classify physiological signals. LS-SVM is a binary classifier. The process of constructing a least squares support vector machine is to solve a quadratic programming problem using the least squares method to find the optimal hyper-plane process that separates two types of training data. The so-called optimal hyper-plane means that the classification surface can not only correctly separate two kinds of data, but also maximize the interval between two kinds of data. When n pairs of data {xᵢ,Yᵢ}i=1^{N} (where xᵢ ∈ Rⁿ is the i^{th} input feature, Yᵢ ∈ R is the corresponding i^{th} category label, i.e. the corresponding seizure state of the EEG signal), the following decision function ƒ(x) can be used to determine its category:

$$f ( x ) = sign [ {\sum }_{i = 1}^{N} {α}_{i} {y}_{i} K ( x , {x}_{i} ) + b ]$$

Where αᵢ is the Lagrange factor obtained from training, ♭ is the classification threshold, and K (x,xᵢ) is the kernel function.

The accuracy of the least squares support vector machine class depends on the quality of the training model. The present invention selects the first-episode EEG data to establish an optimal training model. Firstly, EEG data are processed according to the processes of the above feature extraction, feature sorting and feature selection. The training method is provided as follows: the EEG signal database of epilepsy patients is randomly divided into two parts: 70% and 30%. 70% of the EEG data is used to train the algorithm, and the remaining 30% data is used to test the algorithm so as to obtain a LS-LVM model and related performance indexes.

### Experimental Results

Using this method, an open source EEG database of epilepsy patients from the Department of Epileptology, Bonn University, Germany is used, including 5 subsets, which are labeled Z, O, N, F and S, respectively. Each subset contains 100 equal length EEG signals, each of which is 23.6 s in length and contains 4096 sampling points. Subset Z is collected from 5 healthy individuals with eyes closed, and Subset O is collected from 5 healthy individuals with eyes open. Subset N was collected from the hippocampus of the epilepsy patients. Subset F is collected from the epileptic area of epilepsy patients during the interval period of epilepsy. Subset S is collected from the epileptic area of epilepsy patients during the period of epilepsy. Since it is the most difficult to distinguish EEG during the period of epilepsy and EEG during the interval period of epilepsy, in the present invention, subset S and subset F are selected to test the effectiveness of the method of the present invention. All EEG signals have been marked by epilepsy experts. EEG signals during the interval period of epilepsy are marked as “0” and EEG signals during the period of epilepsy are marked as “1”. This test uses three indexes to evaluate the classification performance, specificity, sensitivity and accuracy. The calculation formula of the three indexes is as follows:

$$Accuracy = \frac{T P + T N}{T P + F N + T N + F P} \times 100 \% , \\ Sensitivity = \frac{T P}{T P + F N} \times 100 \% , \\ Specificity = \frac{T N}{T N + F P} \times 100 \% ,$$

wherein TP, FP, TN and FN respectively represents true positive number, false positive number, true negative number and false negative number.

The EEG data during the period of epilepsy and during the interval period of epilepsy are randomly divided into 70% and 30%, respectively. The least squares support vector machine model is trained and tested for its performance, and compared with other common classification methods. See Table 1 for the specific results. As can be seen from Table 1, the classification results using the method provided by the present invention are optimal.

TABLE 1
Comparison of epileptic EEG signal classification results
between the invention and other 5 common methods

| | Sensitivity | Specificity | Accuracy |
|---|---|---|---|
| Method | (%) | (%) | (%) |
| the method of the invention | 99.50 | 100.00 | 99.40 |
| k-Nearest Neighbor (KNN) | 97.90 | 99.80 | 94.00 |
| Linear Regression (LR) | 99.00 | 100.00 | 98.00 |
| Linear Discriminant | 99.00 | 100.00 | 99.00 |
| Regression (LDA) | | | |
| Naive Bayes (NB) | 91.00 | 98.00 | 84.00 |
| Random Forest (RF) | 97.00 | 99.00 | 9.00 |

EEG signals have important value for epilepsy research. The invention uses a classification system of epileptic EEG signals based on non-linear dynamics features to analyze the EEG signals of epilepsy patients in detail. The sensitivity is 99.50%, the specificity is 100.00%, and the accuracy is 99.40%.

The present invention is not limited to the specific technical solutions described in the above embodiments, and all technical solutions formed by equivalent replacements are the protection required by the present invention.

What is claimed is:

1. A classification system of epileptic EEG signals based on non-linear dynamics features, including a preprocessing module, a feature extraction module, a feature sorting module, a feature selection module, and a classification module, wherein:
the preprocessing module, for preprocessing the EEG signals, uses discrete wavelet transformation (DWT) to remove noise in the EEG data and obtain effective EEG signal data without noise;
the feature extraction module, for dividing the EEG signals into several data segments, uses multiple entropy algorithms to calculate different entropy values of EEG data under the same time window as the characteristic values of the corresponding data segments and a feature set is formed by calculating the entropy values of all entropy algorithms;
the feature sorting module sorts the significant influence on the classification results of epileptic EEG signals according to the entropy values of the extracted EEG signals by use of analysis of variance (ANOVA), and the more significant the influence of feature variables on classification results, the higher the sorting of the feature variables;
the feature selection module uses a forward feature selection (FSFS) algorithm to successively add one feature from the first most significant feature into the classification model until the accuracy of the model is no longer improved, so as to select the optimal feature subset that has the most significant impact on the accuracy of the model; and
the classification module classifies EEG signals of epilepsy patients by use of a least squares support vector machine (LS-SVM) algorithm.

2. The method for classification of epileptic EEG signals based on nonlinear dynamic characteristics according to claim 1, wherein:
the classification module uses the collected EEG signals as training data of a least squares support vector machine to train the classification model; and
the classification model is trained according to the EEG signal database of epilepsy patients, to obtain the hyper parameters of LS-SVM and select an optimized feature subset.

3. The method for classification of epileptic EEG signals based on nonlinear dynamic characteristics according to claim 2, further comprising increasing a real-time on-line system to perform real-time online classification of new EEG signals collected in real-time through the pre-processing module, the feature extraction module, and the classification module.

4. The method for classification of epileptic EEG signals based on nonlinear dynamic characteristics according to claim 1, wherein the discrete wavelet transform method that is used for EEG signal denoising is to use a Daubeches-4 wavelet function, and select an EEG signal with a frequency of 3 to 25 Hz after filtering.

5. The method for classification of epileptic EEG signals based on nonlinear dynamic characteristics according to claim 1, wherein the entropy algorithms are Shannon Entropy, Conditional Entropy, Sample Entropy, and Spectral Entropy.

6. The method for classification of epileptic EEG signals based on nonlinear dynamic characteristics according to claim 2, wherein a training method for the Least Squares Support Vector Machine (LS SVM) algorithm is as follows: randomly dividing the EEG signal database of epilepsy patients into two parts: 70% and 30%, wherein 70% of the EEG data is used to train the algorithm, and the remaining 30% data is used to test the algorithm so as to obtain a LS-LVM model.
